(19) United States
(12) Patent Application Publication
(10) Pub. No.: US 2009/0216250 A1
(43) Pub. Date: Aug. 27, 2009

(54) Device and Method for Carrying Material Through Tissue

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/394,841

(22) Filed: Feb. 27, 2009

Related U.S. Application Data
(60) Provisional application No. 61/067,267, filed on Feb. 27, 2008.

(51) Int. Cl. A61B 17/04

(52) U.S. Cl. 606/144

(57) ABSTRACT

A material carrying device comprising a delivery element having a tapered distal end for penetrating target tissue and a central channel for housing an axially movable carrier element. The delivery element may further comprise an actuating member for both advancing the carrier element to an extended position and withdrawing the carrier element to a retracted position. The carrier element may be composed of any known shape memory material whose native shape will influence the trajectory of the carrier element upon its extension beyond the distal tip of the delivery element. The exit path of the carrier element from the delivery element may also influence the carrier element's trajectory. The scope of the present invention further comprises both a method of carrying material through tissue as well as a providing a shape memory fastener allowing for fixation where only one side of a tissue is accessible.

### CROSS REFERENCE TO RELATED APPLICATIONS

This application claims the benefit of provisional patent application Ser. No. 61/067,267, filed with the USPTO on Feb. 27, 2008, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to memory material devices, more specifically, the present invention relates to both memory material fasteners and memory material devices for passing material through tissues.

2. Background Art

Arthroscopic surgery, laparoscopic surgery, and other less invasive surgical procedures require the suturing of soft tissue in an area not easily accessible due to the lack of space or direction of access to the soft tissue. Existing methods of suturing in such environments create a simple suture knot with only one suture pass through tissue due to the complexity of advancing a suture in such tight access (confined) regions. A mattress suture configuration has been demonstrated as mechanically superior to single pass suturing and has a lower rate of pullout. However, passing a mattress suture (as well as a single pass suture) in less invasive procedures is exceedingly difficult and cumbersome using existing methods, which consist of rigid needles or snares that must be rotated through the soft tissue. A need exists for a device that allows a surgeon to carry suture material through tissue as a means of improving surgical suturing procedures. Such devices and/or methods also provide a benefit to scores of other medical procedures, such as the carrying or passing of treatment slings through bodily tissue. The present inventive device and method provide for such a device and means to carry surgical material through tissue by incorporating memory shape material technology into the carrier element component.

A similar solution is also disclosed for surgical fasteners. Conventional surgical fasteners have resembled staples, barbed pins, and more recently, helical coils. Staple type fasteners require a delivery device which has access to both sides of the tissue. These types of fasteners are hence restricted from use when access to tissue is available only from one side. Barbed pins function like arrow heads. Although they may be utilized in situations where access to tissue may be obtained on one side of tissue, the entry of barbs into tissue inherently damages the tissue. This may compromise the strength of the fixation, and the pin may back out. Both the conventional staple type fastener and the barbed pin rely on barbs to retain tissue or material. Hence, the effective and retentive surface area is limited to the barb structure. This reduced surface area is often sufficient to securely hold the tissue or material.

Helical coils have recently been utilized as fasteners. This type of fastener does place more surface area in contact with tissue or material and also may be utilized when access in only available to one side of the tissue. However, there are still limitations to this configuration. The helical fastener does not place increased surface area in contact with the “attaching tissue or material” (that which is being joined to the target material or tissue). Such attaching tissue or material is often less than 2 mm thick (e.g. mesh). Another limitation of the helical fastener is that it may require deployment from a delivery device that is perpendicular to the receiving tissue or material. Hence, these fasteners cannot be utilized in circumstances which preclude the distal end of a delivery device from making perpendicular or near perpendicular contact with tissue or material.

To circumvent and overcome the problems of conventional fasteners and related applicators, the present invention discloses a simple device that dispenses a surgical fastener that have a high retentive surface area, wherein such fasteners may be deployed at multiple angles other than 90 degrees from the target tissue or other material. Additionally, a device and method incorporating structures beneficially composed of memory shape material technology are disclosed and provide a novel means for carrying material through tissue.

### BRIEF SUMMARY OF THE INVENTION

In accordance with one embodiment, a device for carrying material through bodily tissue comprising a delivery element defining a central channel and having a tapered distal end, a carrier element disposed and axially movable within the central channel of the delivery element, wherein the carrier element comprises a memory shape material wherein the memory shape material is biased to a first shape configuration that is distinct from the shape of the central channel when the carrier element is not disposed within and restrained by the central channel of the delivery element, wherein the strength of said bias of said memory shape material to return to said first shape configuration overcomes at least a portion of an opposition force from said bodily tissue, and an actuating member in communication with the carrier element, wherein the actuating member provides for axial movement of the carrier element within the central channel of the delivery element between a retracted position and an extended position.

Accordingly, it is an object of the present invention to provide surgeons with an easy means of pulling or pushing material through bodily tissue to or from a target location. This device will allow the attachment of material to distant tissue without the need for visualization of a needle tip or tying of a suture. The present inventive device will allow material to be pulled and/or pushed through tissue along pathways not accessible by conventional devices within the art.

Additionally, the scope of the present invention also includes a method for carrying material through bodily tissue comprising the steps of providing a passer device comprising a delivery element defining a central channel, a carrier element disposed and axially movable within the central channel of the delivery element, wherein the carrier element comprises a memory shape material wherein the memory shape material is biased to a first shape configuration that is distinct from the shape of the central channel when the carrier element is not disposed within and restrained by the central channel of the delivery element; the carrier element further comprising a penetrating tip and an adjacent retaining structure, wherein said carrier element creates a pathway through said bodily tissue as said carrier element is advanced from said retracted position to said extended position, and an actuating member in communication with the carrier element, wherein the actuating member provides for axial movement of the carrier element within the central channel of the delivery element between a retracted position and an extended position; advancing the delivery element through the bodily tissue toward a target location; activating the actuating member and motivating the carrier member from the retracted position to the extended position, wherein the strength of the memory shape material of the carrier element is sufficient to form a path through the bodily tissue substantially similar to the first shape configuration of the carrier element as the carrier element is motivated into the extended position; and withdrawing the passer device from the bodily tissue.

### BRIEF DESCRIPTION OF THE DRAWINGS

FIG. 1 depicts a cross sectional side view of an embodiment of a device of the present invention in a retracted state.

FIG. 2 depicts a cross sectional side view of an embodiment of a device of the present invention in an extended state.

FIG. 3 depicts a magnified side view of an embodiment of a handle member of the present invention.

FIG. 4 depicts a magnified perspective view of an embodiment of a handle member of the present invention.

### DETAILED DESCRIPTION OF THE INVENTION

Many surgeries require the advancement, placement, and/or attachment of natural or synthetic material through bodily tissue. Often such tissue is difficult to access or visualize. Although the surgeon may be able to reach the target location with an instrument, it is often quite difficult to attach or place a natural or synthetic material in close proximity to the target location. An even greater challenge is securing the natural or synthetic material to the target location. Present solutions to such surgical difficulties include the use of varying devices that facilitate the passage of surgical suture through distant bodily tissue, devices that place anchors into distant target locations, or large needles that are passed through target bodily tissue.

All of these conventional solutions respectively have their own associated problems. Those that pass a suture through the target tissue require the surgeon to then tie knots in an area that may be extremely difficult to maneuver in or even reach. Those that place anchors in the target tissue may be limited by the geometry of the anchor applicators and are often not suitable for a variety of bodily spaces. Furthermore, the retentive strength of typical soft tissue anchors is substantially less than that of a proper suture. Although large needles may be passed through distant target tissues and thereafter be used to pull the material through the target tissue, the task of attaching the material to the needle may be quite difficult. In such cases, the surgeon must be able to reach the needle tip after it has been passed through the target tissue and the length and geometry of such needles are unfortunately limited. Hence, the surgeon must often choose a target tissue for material attachment that is suboptimal.

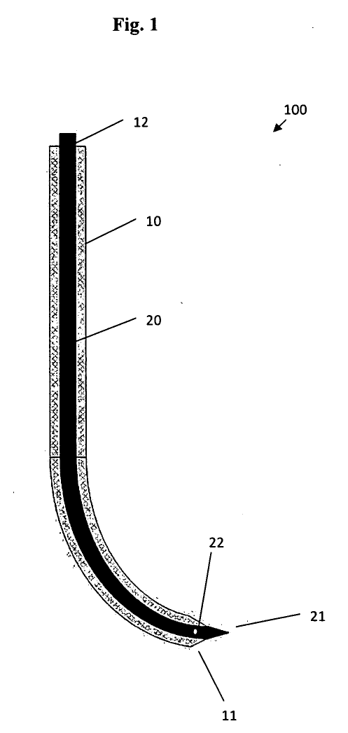

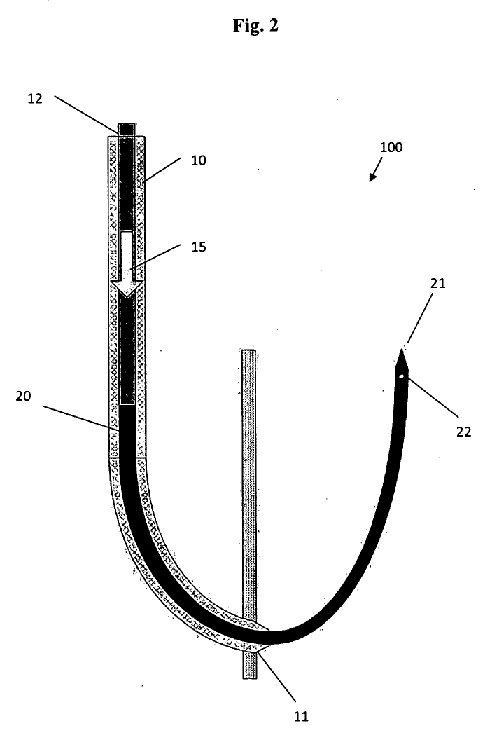

FIGS. 1 and 2 depict a generalized embodiment of a device within the scope of the present invention for passing material through tissue. Such a material passing device 100 may comprise a tubular applicator or delivery element 10 that may have a tapered distal end 11 for penetrating bodily tissue. The delivery element 10 may define a central channel 12 within which a carrier element 20 is disposed and axially movable therein. The delivery element 10 may further comprise an actuating member for providing an advancement force 15 on the carrier element 20 that extends the carrier element 20 through a distal aperture of the delivery element 10 as defined by the tapered distal end 11. Advancement force 15 moves the carrier element 20 from a retracted position (see FIG. 1) to an extended position (see FIG. 2) by any motivating means known within the art. The configuration of the distal portion of the central channel 12 may comprise any number of angles and/or curvatures and may serve to dictate or otherwise influence the exit path and initial trajectory of the carrier element 20 as it exits the tapered distal end 11 of the delivery element 10. While being responsible for providing and advancement force 15 on the carrier element 20, the actuating member may also function to withdraw the carrier element 20 from its extended position (see FIG. 2) to its retracted position (see FIG. 1) by any means known within the art.

The carrier element 20 may comprise an elongated shaft having a distal pointed penetrating tip 21. While in the retracted position, the carrier element 20 is preferably housed entirely within the central channel 12 of the delivery element 10 or the distal penetrating tip 21 of the carrier element may be the only component of the carrier element 20 that extends beyond the tapered distal end 11 of the delivery element 10 (see FIG. 1). In accordance with the present invention, the carrier element 20 may be advantageously composed of Nitinol®, a memory alloy, or any other memory shape material or resiliently shaped material known within the art. In this manner, the carrier element 20 may be restricted to conform to the shape and contour of the delivery element 10 while the carrier element 20 is disposed therein, however, upon exiting the tapered distal end 11 of the delivery element 10 the carrier element 20 has a tendency to return to its first shape configuration or memorized shape with such a tendency being of sufficient strength to overcome at least some of the tissue forces tending to prevent or oppose such a memory-biased trajectory. Thus, a first trajectory influence factor on the carrier element 20 may be the exit path configuration of the delivery element 10 and a second trajectory influence thereafter may be the tendency of the carrier element 20 to create a path conforming to its first shape configuration by means of the memory shape characteristics influencing the carrier element 20 as it exits the distal end 11 of the delivery element 10.

For a surgeon to be able to anticipate the overall trajectory of the carrier element 20 during its advancement, it is highly beneficial to prevent rotation of the carrier element 20 disposed within the delivery element 10. Any such rotation will dramatically reduce the accuracy of the carrier element's 20 projected or estimated penetrating trajectory. To eliminate the possibility of such rotation, the shape of the carrier element 20 may be such that an interface with a complimentarily shaped central channel 12 of the delivery element 10 prevents rotation. As an illustrative example, a ribbon-shaped carrier element 20 advancing through a central channel 12 having a complimentary shaped rectangular cross section within the delivery element 10 would effectively restrict all relative rotation between the two elements. Additionally, complimentary structures such as a peg extending from the surface of the carrier element 20 may slide within a complimentary shaped slot or groove disposed axially along the inner wall of the delivery element 10. In this manner, any form of such complimentary structures known within the art may be incorporated into the device 100 to restrict rotation between both the delivery element 10 and the carrier element 20 be improve the accuracy of the estimated trajectory of the carrier element 20. Yet further, the carrier element 20 may be directly attached to a portion of the delivery element 10 to prevent rotation of the carrier element 20.

Both the curvature and/or angles of the carrier element 20 and the biased first shape configuration conformed to outside of the delivery element 10 may be specifically selected by the surgeon on a case-by-case basis to achieve the estimated or projected trajectory necessary for the particular surgery. To improve the probability of the memory shape material cutting a path in accordance with its biased native shape, the distal penetrating tip 21 of the carrier element 20 may be tapered to allow the carrier element 20 to more easily penetrate soft tissue while advancing 15 along its biased shape memory trajectory path. The distal penetrating tip 21 of the carrier element 20 may have at least one retaining structure 22 including but not limited to eyelets, protuberances, defects, and the like, which allow for the attachment of material to the distal tip 21 of the carrier element 20. In this manner, any necessary material may be attached to the carrier element 20 after an advancement force 15 has motivated the carrier element 20 into its extended position (see FIG. 2). Alternatively, the necessary surgical material (e.g. a suture, sling, or other surgical material) may be attached to the at least one retaining structure 22 so that advancement of the carrier element 20 transports the surgical material from the initial insertion point to the target location.

In use and as depicted in FIG. 1, the carrier element 20 is initially placed into a retracted position prior to insertion through an accessible side of the bodily tissue. The surgeon may then insert the tapered distal end 11 of the delivery device 10 through the bodily tissue. The surgeon then activates the actuating member that provides an advancement force 15 on the carrier element 20 and advances the carrier element 20 through and beyond the distal end 11 of the central channel 12 of the delivery element 10. In this manner, the distal penetrating tip 21 of the carrier member 20 is advanced into and through the bodily tissue (see FIG. 2). The memory shape material of the carrier element 20 and the angle of exit of the carrier element 20 from the delivery element 10 assist in influencing the path of the carrier element 20. The surgeon advances the carrier element 20 along its anticipated trajectory, creating a pathway in the tissue with the carrier element 20, until he or she is able to more easily access the distal penetrating tip 21 of the carrier element 20 at a target location. The surgeon may then attach a material necessary for the particular surgical procedure onto at least one retaining structure 22 at the tip 21 of the carrier element 20. The surgeon then activates the actuating member to withdraw or return the carrier element 20 through the path it created to its initial retracted position within the delivery element 10. This brings the attached surgical material back through the pathway of the carrier element 20. The delivery element 10 may then be withdrawn from the bodily tissue, bringing the attached material back through the pathway of the delivery element 10. Finally, the attached material may then be separated from the carrier element 20 and is readily accessible to the surgeon for further manipulation. The angulated paths created by the differing trajectories of the delivery element 10 and carrier element 20 leave the material an overall pathway that provides substantial retentive and resistive forces that may obviate the need for further securing the material.

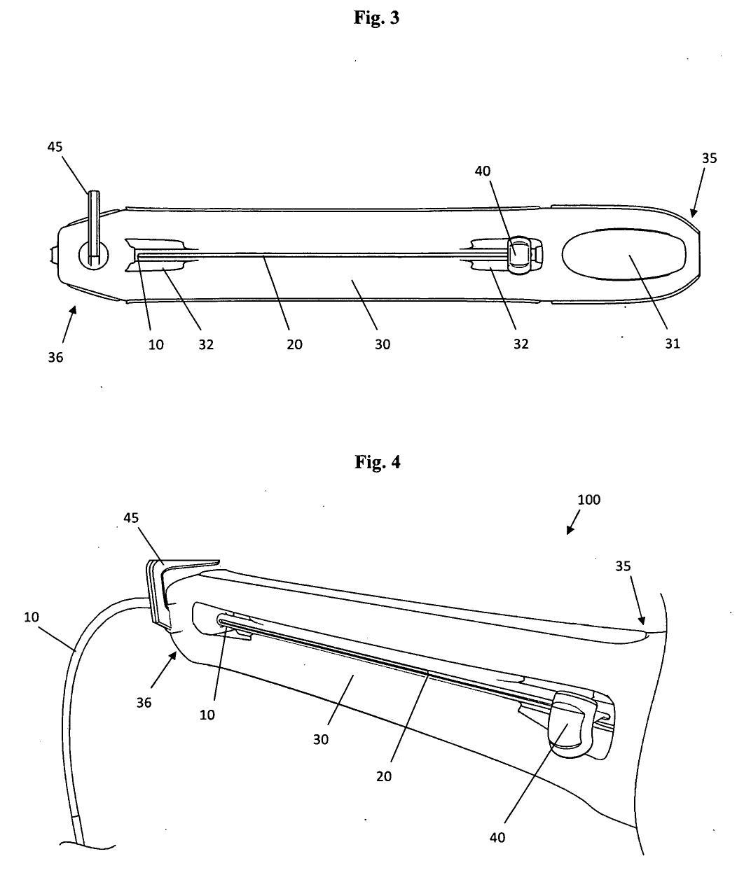

FIGS. 3-4 depict a side view and a perspective view, respectively, of another embodiment of the present invention having a highly functional handle member 30. FIGS. 3-4 illustrate a magnified view of the handle member 30 and general proximal end 35 of the device 100. The delivery element 10 may enter the distal end 36 of the handle member 30 and be accessible via at least one handle aperture 32 disposed within the handle member 30. The actuating member 40 may communication with the carrier element 20 through the at least one handle aperture 32. Actuating member 40 provides for axial translation of the carrier element 20 within the delivery element 10. In one embodiment, an articulating member 45 may be used to flex the delivery element 10 further altering the trajectory of the delivery element 10. The actuating member 40 may remain in a biased locked configuration, wherein depression of the actuating member 40 releases the actuating member 40 and allows for axial translation of the carrier member 20 while depression of the actuating member 40 is maintained. The handle member 30 may further comprise one or more manual grips 31 to assist in the grasping and manipulation of the device 100 and the handle portion thereof.

The scope of the present invention further comprises a method for delivering and/or retrieving material though tissue or other material along a path that may have more than two curves or angles, three curves or angles, four curves or angles, or a plurality of curves or angles. In this method the surgeon approaches the target location using a primary delivery element 10 to create the primary path. Then the surgeon uses the primary delivery element 10 to deliver a carrier element 20 made of a memory shape material capable of forging or penetrating tissue through a path of a different trajectory than that of the primary delivery element 10. This process may be repeated any number of times via the addition of a plurality of delivery elements 10 and associated carrier elements 20 that each serve to further extend the path and increase the overall number of respective curves or angels disposed along the path.

All conventionally known methods of creating paths for the delivery or retrieval of materials are limited by the shape of the primary delivery element 10. This primary delivery element 10 typically houses a carrier element 20 or needle that may be advanced to further extend the path being created. Such a path would typically continue along the trajectory of the primary delivery element or its exit channel. The present inventive device and method overcome this limitation. The present inventive device and method are uniquely capable of creating a path that is not limited to the trajectory of the primary delivery element or its exit channel. Additionally, the present invention further utilizes a carrier element 20 comprised of memory shape material having a shape memory bias that is of sufficient strength to penetrated tissue along a trajectory substantially similar to the native shape conformation possessed by the memory material. In other words, the tendency for the memory shape material to return to its native, first shape configuration, or resting configuration is greater than the tendency of the surrounding tissue to prevent such movement and/or the tendency of the memory shape material to return to its resting position is of sufficient strength to overcome at least a portion of the tissue forces tending to prevent such movement of the carrier element 20.

When access is available to both sides of a piece of tissue, a fastener or anchor may be deformed as it exits the tissue (e.g. a staple). This deformation increases the retentive strength and/or resistance of the fastener to pull out forces. When access to both sides of a piece of tissue is not possible, fasteners must be delivered in their final shape. Hence, presently there are great limitations imposed on the available geometry of such fasteners where access is only available to one side of the tissue. Conventional fastener and/or anchor shapes include tacks both with and without barbs, screws, and helixes. No presently used fasteners or anchors substantially change shape either inside the delivery device or inside the tissue during deployment. The scope of the present invention includes a fastener composed of memory shape metal or other memory material known within the art that conforms to the shape of its delivery device and then changes shape back to its native form upon exit from the delivery device. The change in shape will inherently be toward the original native shape of the memory shape fastener (e.g. shape configuration prior to its confinement within the delivery device).

The present invention comprises a surgical fastener and a delivery device for delivering the fastener into body tissue or other target location. The fastener and delivery device of the present invention may be used in a wide variety of medical procedures including but not limited to ligation procedures, hernia mesh repairs, other hernia repairs, prolapse surgery, incontinence surgery, in conjunction with drug delivery systems and surgical implantable devices, and the like.

The preferred embodiment of a surgical fastener of the present invention may be that of a continuous or non-continuous coil or ring which deforms temporarily to a shape approximating that of its delivery device when disposed therein. The fastener comprises a biased tendency toward regaining its original memory shape when the fastener is deployed from within the delivery device. Hence, the fastener must be manufacture from resilient material and/or any material having memory shape properties or characteristics. Such materials may be either permanent or absorbable within the bodily tissue. Once deployed, the fastener's pre-disposed memory shape bias has a preferable tendency to physically bind or connect itself between a tissue and a material, a tissue and a tissue, or a material and a tissue. The biased, memory shape characteristics of the present inventive fastener allow it to be deployed in a direction not in axial alignment with the main body of the delivery device (e.g. the fastener may travel a curved path to exit the delivery device) and to enter bodily tissue or a target location at any angle between 1 degree to 90 degrees.

The present inventive fastener may be configured in a multitude of shapes or embodiments. These shapes may include but are not limited to straight, curved, coiled, angled geometries, and any combinations thereof. The fastener may further be created with or without barbs, protuberances, and the like. The distal end or ends of the present fastener may be tapered or sharpened to facilitate penetration into bodily tissue or other material. Additionally, the present inventive fasteners may be comprised of a solid core of memory shape material or, as an alternate embodiment; the fasteners may comprise memory shape material having an internal hollow or channel at their core. The proximal end of the fastener may have features that act to bind or capture bodily tissue or other material.

The applicator or delivery element may comprise but is not limited to two preferred embodiments. One such delivery element embodiment delivers present inventive fasteners having pointed or sharp end(s) that are independently capable of penetrating bodily tissue or other material. The second delivery element embodiment of the present invention may deliver fasteners that lack sharp or pointed end(s) and need additional assistance in penetrating bodily tissue or other material. Such an embodiment may further comprise fasteners having an internal hollow or channel therein.

A first embodiment a fastener delivery element may comprise a linear, curvilinear, or curved tube having an inner plunger for delivering an axial or longitudinal force against the proximal end of a fastener. The distal, dispensing end of the delivery element may have phalanges, protuberances, or other structures for holding or carrying bodily tissue or other material. The device may have a first configuration allowing for the loading of single fasteners at a time and a second configuration allowing for the loading of multiple fasteners at a time. The devices may be calibrated to dispense a single fastener per activation. The device may be composed of disposable or reusable material that may be sterilized for repeated use. The device may incorporate an internal feature or structure for maintaining the necessary alignment of the fastener that is required to maintain proper fastener orientation for ejection and deployment. This feature or structure may be in the form of a groove within the device that receives a protuberance or feature of the fastener or any other such means known within the art. Orientation of the fastener may be assisted by the specific complimentary shape of the internal dispensing chamber of the delivery element to the fastener, thus preventing rotation of the fastener while it is disposed within the dispensing chamber. Alternatively, the plunger element may prevent rotation of the fastener via the manner of physical communication in which the plunger may contacts the fastener.

A second embodiment may comprise a delivery element for delivering fasteners, wherein the delivery element comprises a hollow or internal channel therein. The main body of the delivery element may comprise a linear, curvilinear, or curved tube having a central or offset pin. The pin may run into the hollow or internal channel of the fastener. When fabricated for the delivery of fasteners whose internal channel communicates with both ends of the fastener, multiple fasteners may be loaded into or onto the delivery device. When fabricated to deliver fasteners with dull distal ends (not capable of penetrating tissue) the distal end of the device may serve as the penetrator.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the examples given.

What is claimed is:

1. A device for carrying material through bodily tissue comprising:
a delivery element defining a central channel and having a tapered distal end;
a carrier element disposed and axially movable within said central channel of said delivery element, wherein said carrier element comprises a memory shape material wherein said memory shape material is biased to a first shape configuration that is distinct from the shape of said central channel when said carrier element is not disposed within and restrained by said central channel of said delivery element, wherein the strength of said bias of said memory shape material to return to said first shape configuration overcomes at least a portion of an opposition force from said bodily tissue; and
an actuating member in communication with said carrier element, wherein said actuating member provides for axial movement of said carrier element within said central channel of said delivery element between a retracted position and an extended position.

2. The device of claim 1, wherein said tapered distal end of said delivery element influences the trajectory of said carrier element as said carrier element advances beyond said tapered distal end of said delivery element and retakes said first shape configuration.

3. The device of claim 2, further comprising:
an articulating member for flexing said delivery element, wherein said articulating member thereby alters said trajectory of said carrier element.

4. The device of claim 1, wherein said carrier element comprises a penetrating tip to facilitate the piercing of said bodily tissue, wherein said carrier element creates a carrier path through said bodily tissue as said carrier element is advanced from said retracted position to said extended position.

5. The device of claim 1, wherein said carrier element further comprises:
a retaining structure for securing said material to said carrier element.

6. The device of claim 5, wherein said retaining structure is selected from the group consisting of an eyelet, a protuberance, and a defect.

7. The device of claim 1, further comprising:
a handle member disposed about at least a portion of said delivery element.

8. The device of claim 7, wherein said handle member further comprises at least one handle aperture providing access to said carrier element within said handle member.

9. The device of claim 1, wherein said actuating member is biased into a locked position thereby preventing said axial movement of said carrier element and depression of said actuating member moves said actuating member into an unlocked position and allows for said axial movement of said carrier member while said actuating member is depressed.

10. The device of claim 1, wherein said first shape configuration of said carrier element comprises at least one curve or angle.

11. The device of claim 1, wherein said first shape configuration of said carrier element is straight or linear in shape.

12. The device of claim 1, wherein said strength of said bias of said memory shape material of said carrier element forms a carrier path through said bodily tissue substantially similar to said first shape configuration of said carrier element as said carrier element is motivated into said extended position.

13. A device for carrying material through bodily tissue comprising:
a delivery element defining a central channel and having a tapered distal end;
a carrier element disposed and axially movable within said central channel of said delivery element, wherein said carrier element comprises a memory shape material wherein said memory shape material is biased to a first shape configuration that is distinct from the shape of said central channel when said carrier element is not disposed within and restrained by said central channel of said delivery element, wherein the strength of said bias of said memory shape material to return to said first shape configuration overcomes at least a portion of an opposition force from said bodily tissue;
a handle member disposed about at least a portion of said delivery element;
an actuating member in communication with said carrier element, wherein said actuating member provides for axial movement of said carrier element within said central channel of said delivery element between a retracted position and an extended position; and
an articulating member for flexing said delivery element, wherein said articulating member thereby alters the trajectory of said carrier element;
wherein said strength of said bias of said memory shape material of said carrier element is sufficient to form a carrier path through said bodily tissue substantially similar to said first shape configuration of said carrier element as said carrier element is motivated into said extended position.

14. The device of claim 13, wherein said actuating member is biased into a locked position thereby preventing said axial movement of said carrier element and depression of said actuating member moves said actuating member into an unlocked position and allows for said axial movement of said carrier member while said actuating member is depressed.

15. A method for carrying material through bodily tissue, comprising the steps of:
providing a passer device comprising:
a delivery element defining a central channel;
a carrier element disposed and axially movable within said central channel of said delivery element, wherein said carrier element comprises a memory shape material wherein said memory shape material is biased to a first shape configuration that is distinct from the shape of said central channel when said carrier element is not disposed within and restrained by said central channel of said delivery element; said carrier element further comprising a penetrating tip and a retaining structure, wherein said carrier element creates a carrier path through said bodily tissue as said carrier element is advanced from a retracted position to an extended position; and
an actuating member in communication with said carrier element, wherein said actuating member provides for axial movement of said carrier element within said central channel of said delivery element between said retracted position and said extended position;
advancing said delivery element through said bodily tissue toward a target location forming a delivery path;
activating said actuating member thereby motivating said carrier member from said retracted position to said extended position, wherein the strength of said memory shape material of said carrier element forms said carrier path through said bodily tissue substantially similar to said first shape configuration of said carrier element or at least substantially different from said delivery path of said delivery element as said carrier element is motivated into said extended position; and
withdrawing said passer device from said bodily tissue.

16. The method of claim 15, wherein the trajectory of said carrier path created by said memory shape material of said carrier element is distinct from the trajectory and said delivery path created by said delivery element.

17. The method of claim 15, wherein said passer device further comprises:
an articulating member for flexing said delivery element, wherein said articulating member thereby alters the trajectory of said carrier element.

18. The method of claim 17, further comprising the step of.
activating said articulating member thereby flexing said delivery element and altering said trajectory of said carrier element.

19. The method of claim 15, wherein prior to said advancing of said delivery element through said bodily tissue said material is secured to said retaining structure of said carrier element thereby providing for passage and delivery of said material to said target location.

20. The method of claim 15, wherein prior to said withdrawing of said passer device from said bodily tissue said material is secured to said retaining structure of said carrier element thereby providing for passage and retrieval of said material from said target location.

21. The method of claim 15, wherein said advancing of said delivery element and said activating of said actuating member occur through said delivery path and said carrier path having more than one curve or angle, more than two curves or angles, more than three curves or angles, or more than four curves or angles.
